(19) United States
(12) Patent Application Publication
(10) Pub. No.: US 2002/0082573 A1
(43) Pub. Date: Jun. 27, 2002

(54) Liquid absorbing pad and method of manufacturing the same

(76) Inventor: Loretta Maureen McGrath Hill, Detroit, MI (US)

Correspondence Address: Dinnin & Dunn, P.C., Ste. 2100, 755 West Big Beaver, Troy, MI, 48084, US

(21) Appl. No.: 09/997,109

(22) Filed: Nov. 29, 2001

Related U.S. Application Data
(60) Provisional application No. 60/250,028, filed on Nov. 29, 2000.

(51) Int. Cl. A61F 13/15

(52) U.S. Cl. 604/366

(57) ABSTRACT

A disposable medical pad comprising a perforated top layer, a perforated intermediate layer proximate to said top layer, said intermediate layer comprising a plurality of sealed and at least partially inflated chambers, an absorbent layer proximate to said perforated intermediate layer and a bottom non-porous layer which forms a tray to retain the waste material.

Continuation of Provisional Patent Application 60/250,028—Filed: Nov. 29. 2000

### BACKGROUND OF THE INVENTION

The instant invention relates generally to a bed pad and specifically to a pad that protects the bony prominence of the human body and allows waste material and liquids to be removed from contact with sensitive skin tissue, thereby preventing skin breakdown from chronic exposure to bodily secretions and excretions. Furthermore, the device protects the surface of a bed, chair seat, crib, linens, or other objects from becoming soiled, thereby enhancing hygienic conditions in medical facilities and home care sites. The invention also relates to the cost-effective manufacture of the device in a manner that it can be readily used in medical facilities and home care sites.

Skin breakdown is a serious medical problem. The body constantly secretes fluids and waste materials, particularly from wounds. The build-up of waste materials and moisture proximate skin tissue causes the skin to tear and to breakdown relatively quickly.

Many elaborate and expensive devices have been used to minimize the collection of waste materials next to the skin and the attendant breakdown of the skin. Once skin integrity is compromised, infection and deterioration of the skin occur rapidly, leading to patient discomfort, a deterioration in the patient's health and requiring further medical treatment. This chronic problem costs the health care industry billions of dollars every year. Many lives are lost because of this problem. The elderly, handicapped and neonatal newborns are especially vulnerable due to their relative immobility. Many individuals temporarily immobilized due to surgery or trauma are also vulnerable to skin breakdown. Furthermore, the collection of liquid and solid bodily wastes from patients provides a constant risk of exposure to health care workers.

Prior art attempts to solve the above-mentioned problems include air mattresses. Air mattresses do provide the type of cushioning which improves comfort for bed-ridden or sedentary patients. However, air mattresses require non-porous material to retain the air which pads the patient and thus are cumbersome, hot, and occlusive and cause moisture and waste material to collect in low spots. If these air mattresses are not frequently changed, their surface becomes contaminated rapidly. Furthermore, air mattresses require inflation, are difficult to secure bed linens to, are easily punctured by sharp objects such as needles and other medical devices, and are quite expensive to produce and use.

Very expensive flex-air bed mattresses area available which circulate air through a fabric material. While this provides increased comfort and reduces collection of waste next to the skin, the costs associated with these types of beds and mattresses are such that their use is limited to a select few patients.

### SUMMARY OF THE INVENTION

The instant invention is an inexpensive disposable absorbent pad that efficiently allows moisture and waste products to drain away from a patient's skin. The liquid absorbing pad comprises a plurality of material layers for removing moisture and solid wastes and a novel padding layer of three-dimensional interconnected cells containing a pressurized liquid or gas. The cells support the weight of the patient or of a body part and provide a barrier between the patient and the drained waste materials. The cells can have any three dimensional shape capable of efficient pressurization that allows for a plurality of interstices therebetween when the cells are arranged adjacent one another. An impervious bottom layer having walls that enclose and support the pad provides a reservoir for waste materials.

The liquid absorbing pad of the instant invention can be manufactured from readily available inexpensive materials by primary manufacturing technique in the form of a roll of disposable material with multiple perforation points. This allows the user to size the pad for the particular application required. For example, if an arm or leg wound requires wrapping, a relatively short section of the pad can be removed from the roll and secured to the wound site. Alternatively, a longer section of the pad can be utilized for complete bed protection. Once soiled, the pad is simply discarded.

### BRIEF DESCRIPTION OF THE DRAWINGS

FIG. 1 is an exploded isometric view of a liquid absorbing pad in accordance with the instant invention.

FIG. 2 is a plan view of a surface barrier layer in accordance with the instant invention.

FIG. 3 is a partial isometric view of the instant invention.

FIG. 4 is a partial isometric view of the instant invention.

FIG. 5 is a plan view of a padding layer in accordance with the instant invention.

FIG. 6 is a view of a suggested application of the instant invention.

FIG. 7 is a view of a suggested application of the instant invention.

FIG. 8 is a view of a suggested application of the instant invention.

FIG. 9 is a view of a suggested application of the instant invention.

FIG. 10 is a view of a suggested application of the instant invention.

FIG. 11 is a view of a suggested application of the instant invention.

FIG. 12 is an isometric view of a step in the preferred method of manufacturing a liquid absorbing pad.

FIG. 13 is an isometric view of a step in the preferred method of manufacturing a liquid absorbing pad.

FIG. 14 is an isometric view of a step in the preferred method of manufacturing a liquid absorbing pad.

FIG. 15 is an isometric view of a step in the preferred method of manufacturing a liquid absorbing pad.

FIG. 16 is an isometric view of a step in the preferred method of manufacturing a liquid absorbing pad.

### DETAILED DESCRIPTION OF THE PREFERRED EMBODIMENT

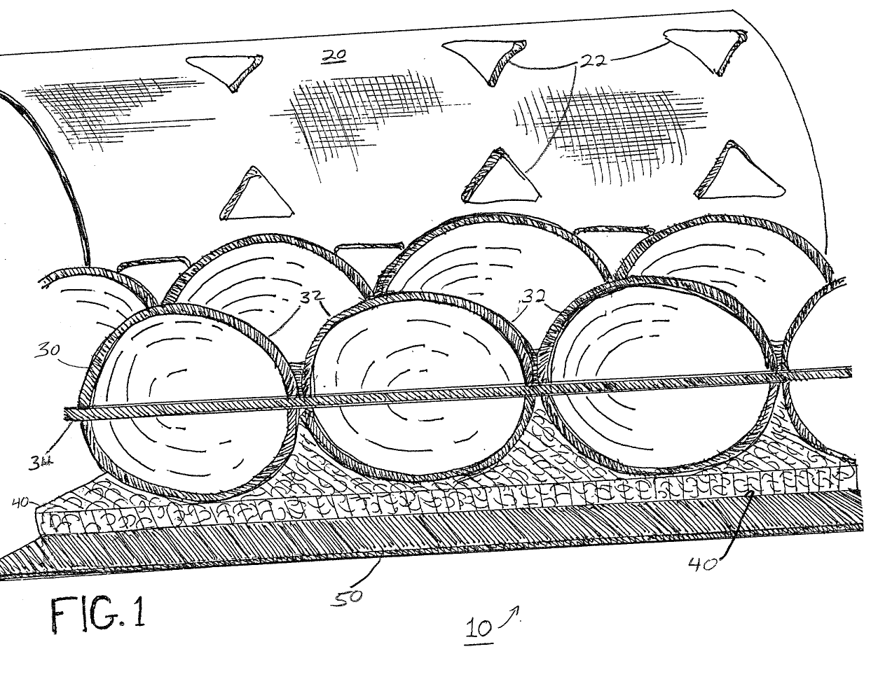

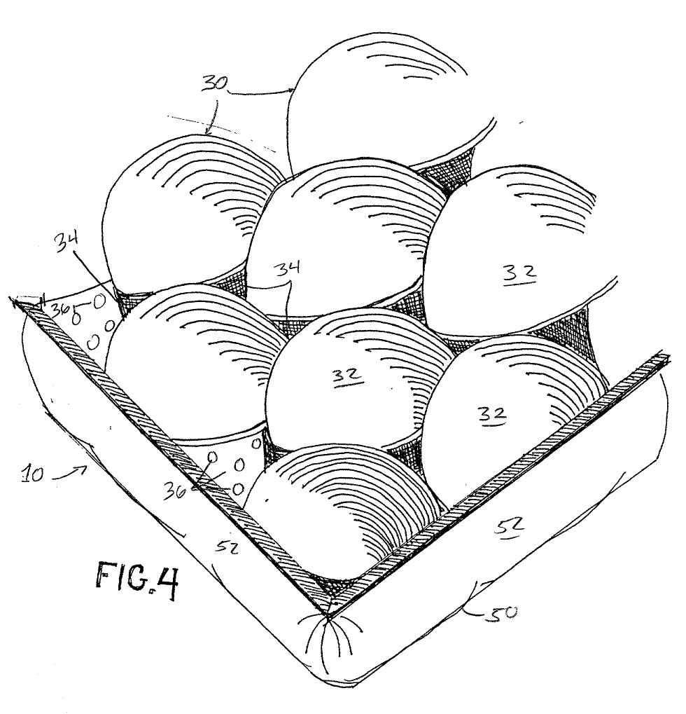

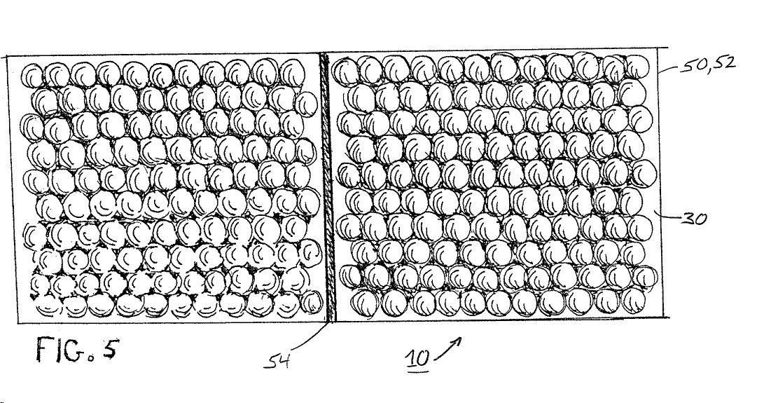

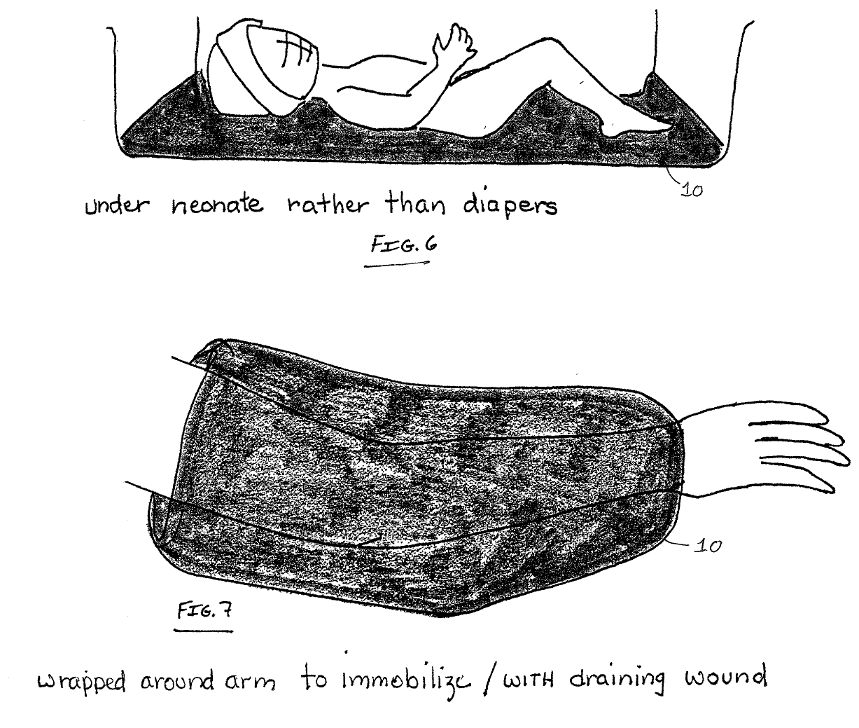

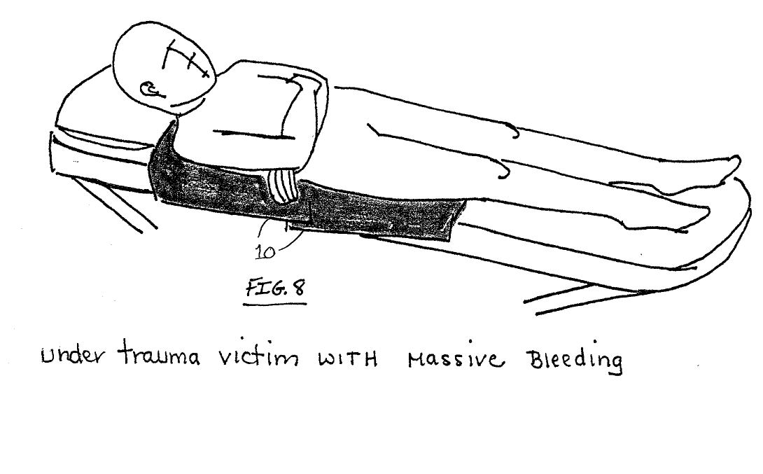

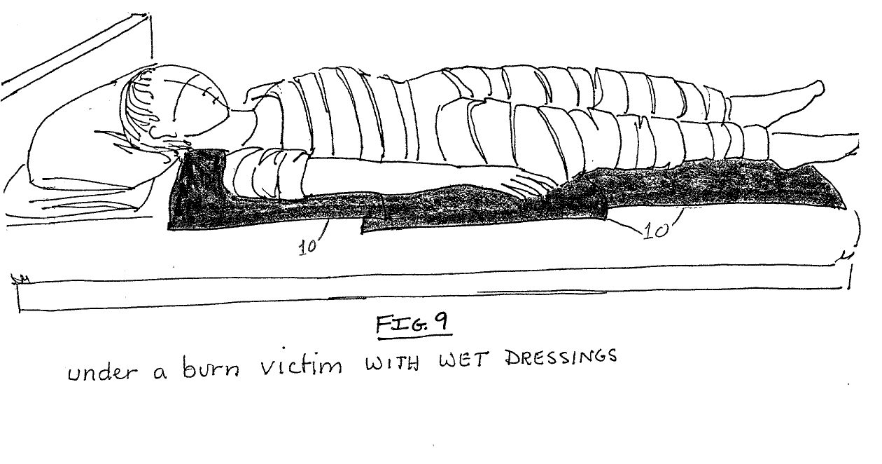

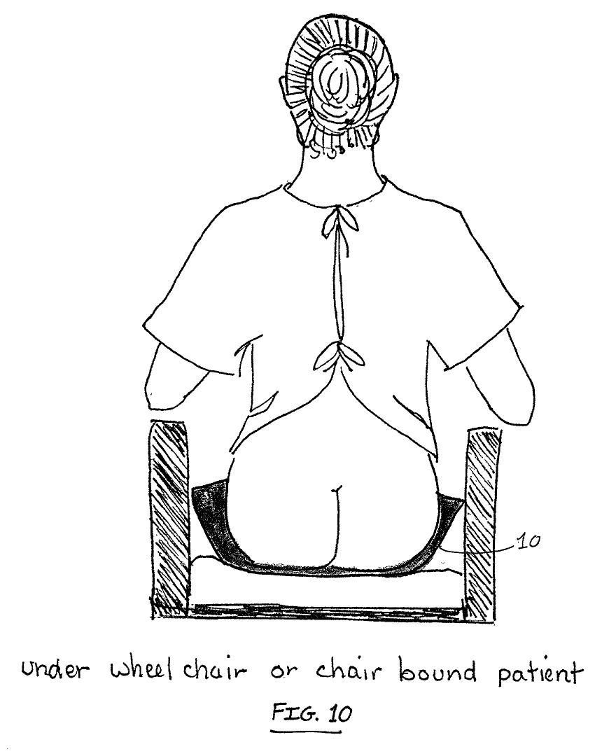

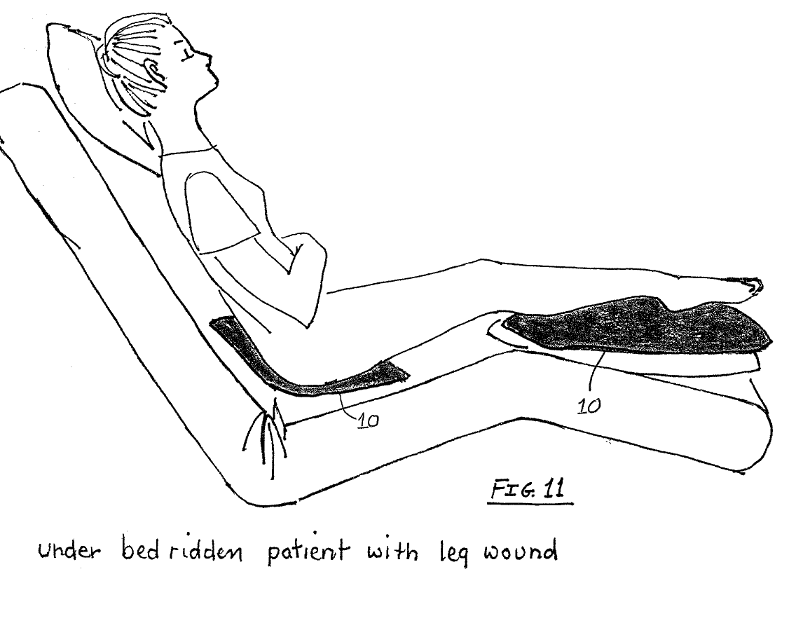

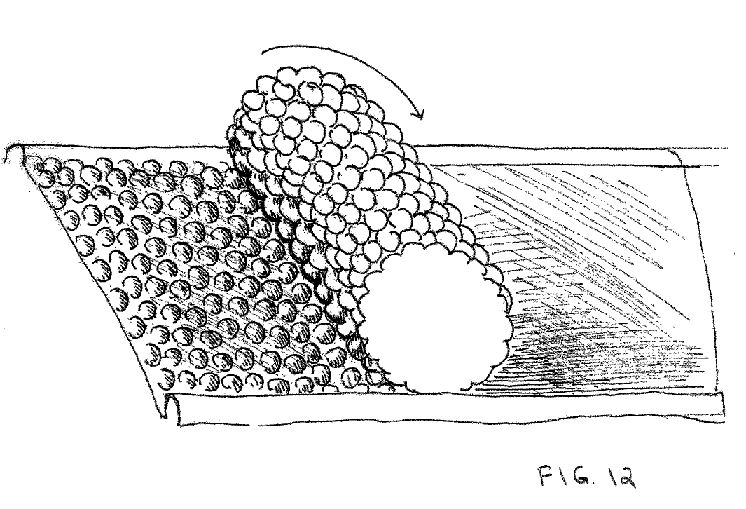

As shown in FIGS. 1, 2, 3 and 4, a liquid absorbing pad 10 in accordance with the preferred embodiments of the instant invention comprises a surface barrier layer 20 of non-absorbent material, a padding layer 30, a layer of absorbent paper 40, and a bottom impervious protective layer 50 having integral walls 52, thereby forming a reservoir around the aforementioned pad layers.

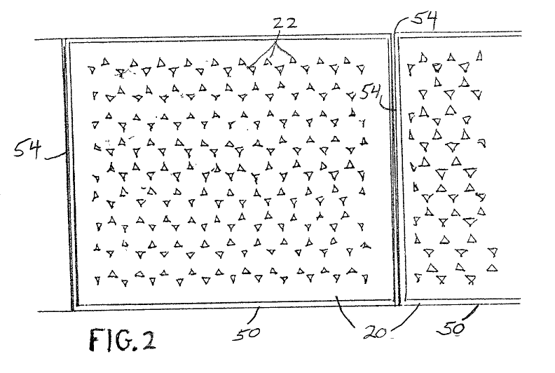

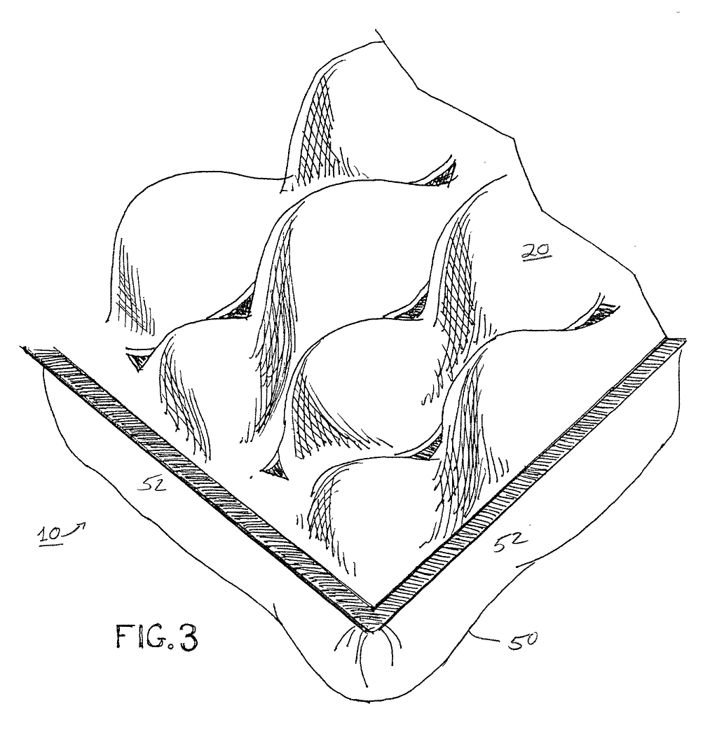

As best seen in FIGS. 1 and 2, the surface barrier layer 20 is preferably comprised of a nonabsorbent, pervious material that allows moisture and waste materials to pass therethrough into the depth of the pad. This surface layer 20 has a plurality of spaced perforations or apertures 22 therein to enhance waste flow into the next layer of the pad 10. The surface barrier layer 20 is preferably comprised of a fibrous non-absorbent material, such as paper, to allow moisture to pass into the pad 10 without excessive contact with a patient's skin. The plurality of apertures 22 in the barrier layer 20 also allows air to circulate around the patient's skin. Furthermore, and as best seen in FIG. 3, the surface layer 20 of the instant invention may be convoluted, to further enhance airflow to the patient's skin.

Below the surface barrier layer 20 is disposed a padding layer 30 comprised of a plurality of individual three-dimensional cells 32 having a liquid or gas therein, disposed adjacent one another and connected by an integral membrane 34 having a plurality of apertures 36 therein. The plurality of cells 32 is under sufficient pressure so as to accept the weight of a patient or body part without completely flattening. Some compression of individual cells is preferred, allowing the pad 10 to conform to an extent to the contour of the patient, or portion thereof supported by the pad. Since the cells 32 are disposed adjacent each other a patient's weight is distributed over the padding layer 30 as a whole. The cells 43 are preferably comprised of a vinyl or plastic material that allows each cell 32 to be flexible yet resistant to puncture. In one embodiment of the instant invention the plurality of cells 32 is manufactured from bubble wrap material, as is used in shipping and packaging applications. In a preferred embodiment of the instant invention the cells 32 are preferably spherical in shape and one to one and one-half inches in diameter, but can be any three-dimensional shape that can be arranged to provide a plurality of interstices therebetween.

The membrane 32 may be compromised of the same flexible material as the cells 32 and bisects each adjacent cell 32 at approximately its midline, as best seen in FIG. 2. The membrane 34 is used to connect the adjacent cells 32 to form the padding layer 30. Furthermore, the membrane 34 has a plurality of apertures 36 therein to allow waste materials to pass through. This feature of the instant invention removes waste material further from the patient and collects it, as will be discussed herein.

Below the padding layer 30 is disposed a moisture absorbent paper layer 40, for example similar to that employed in conventional disposable diapers. This material absorbs moisture passing through perforations in the membrane in the padding layer 30, thereby prohibiting it from contact with the patient.

A protective non-porous layer 50 is disposed below the moisture absorbent paper 40, comprised of a thin layer a vinyl or plastic. In a preferred embodiment of the instant invention the protective layer 50 is similar in thickness to the outer layer of a conventional disposable diaper. The protective layer 50 is slightly larger in area that the padding layer 30 thereabove, to allow the pad 10 to be rolled efficiently, thus promoting ease of shipping and storage. Furthermore, the vinyl protective layer 50 has vertically oriented walls 52 that protect and surround the other pad 10 layers. The protective layer 50 is heat bonded around its edges to provide a tray or reservoir for the collection of waste without the waste leaking out the edges of the pad. This feature of the instant invention allows the pad 10 to collect large quantities of liquid waste prior to being changed, and provides for easy disposal without contamination of surroundings. Furthermore, since the materials necessary to manufacture the pad 10 are readily available and inexpensive, the pad 10 is disposable. This greatly reduces costs to hospitals and long-term care facilities by freeing staff from the time consuming task of laundering bed pads.

In a preferred embodiment of the instant invention, a continuous roll of pad 10 is divided into a plurality of sections connected by a vinyl perforation 54 so that the user may select an appropriate pad size by simply separating a section of pad 10 from the roll at a perforation point.

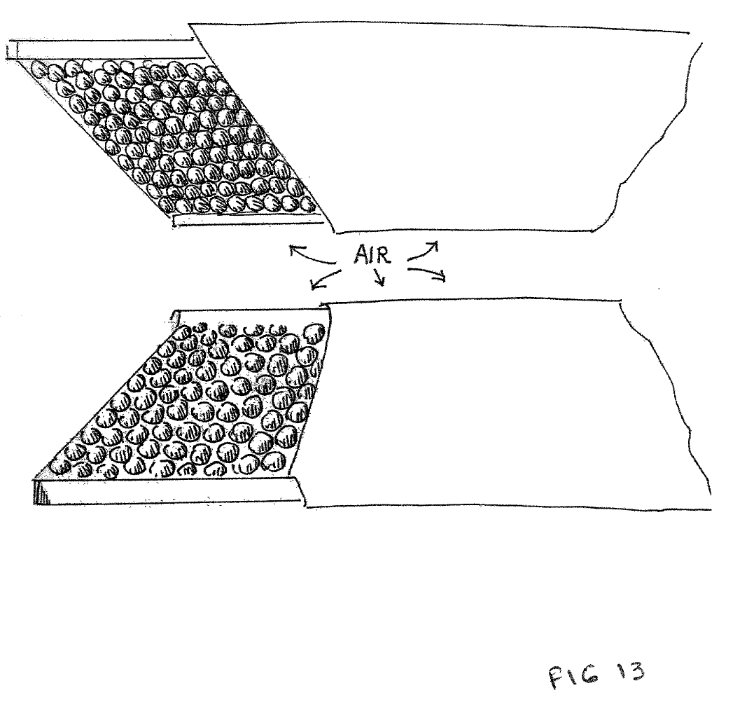

The device according to the present invention can be manufactured in a relatively cost effective manner and so that it can be readily packaged for shipping and for user storage and use. While the system is similar to the known method for the production of “bubble wrap”, the preferred method includes steps which are not believed to have been used in such systems as described herein due to the unique nature of the combination of elements. The first step illustrated, which is preferably first if an assembly process if utilized, is the processing of the top layer which preferably conforms to the bubbles to be formed in the intermediate layer as shown in FIG. 12. This is achieved by passing the layer 20 beneath a stationary roller 60 having protrusions 62 matching the die indentations of the next step. If desired, the strip could remain stationary and the roller advance across the strip to create the indentations.

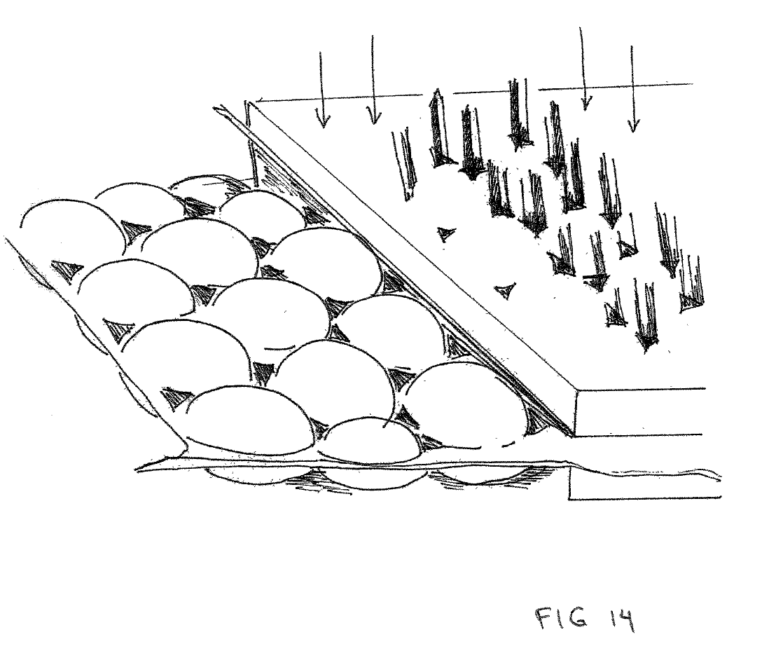

As shown in FIG. 13, the top layer 20 is then advanced to the “bubble” station and between opposing dies containing indentations. Although a variety of shapes can be used, such as channels or squares, semi-spherical shapes are believed to provide the most padding at the lowest material cost within a given volume; this also allows for a uniform distribution of drain perforations to promote draining. Preferably, the indentations formed in the top layer match the die indentations so that the top layer can be aligned within the die and will conform with the lower die surface. Two sheets of thermoset plastic 64, 66 are placed or fed on top of the top layer, and air blown in between the layers. The dies are closed and heated, so that the die surfaces surrounding the matching semi-spherical indentations will meet and bond the thermoset layers therebetween to form spherical chambers or “bubbles”. The air flowing between the sheets will urge the plastic layers apart to conform to the indentations as the die are brought together, and as the plastic is heated, it will tend to flow to conform to the shape of the heated die surfaces. To allow for the deformation of the plastic layers, the sheets are preferably chosen to be greater in width than the die and thicker than necessary in the resultant product. The plastic layers are also selected to be wide enough to form an edge or lip 38 formed by flat heated lateral edges of the dies as they meet.

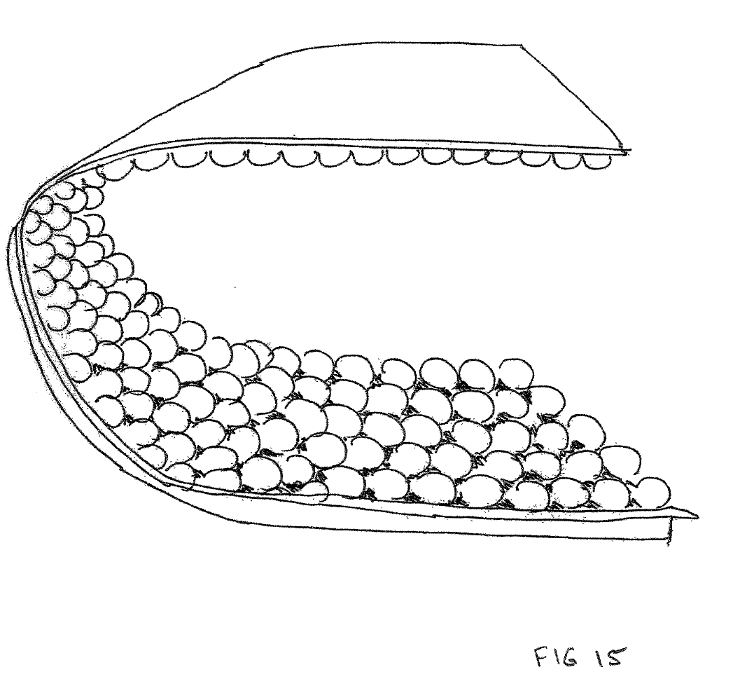

The top layer and newly formed intermediate padding or “bubble” layer are preferably perforated simultaneously to allow waste material to drain. The perforations are formed, preferably in the chamber or bubble forming stage, by pressing a plurality of spikes 68 or heated rods through matching apertures in the flat die surface as shown in FIG. 14. In this manner, the air chambers or bubbles will not be punctured and the perforations can be accurately located. The heated rods will melt holes in the plastic which will cause the plastic to flow around the perforations, thus strengthening the resulting layer around the perforations and dispensing with chad that would result from stamping or punching the perforations. In these forming steps, the top layer may be adhered to the bubble layer or included in the heat bonding.

It is also preferred to form a regular series of perforations to allow a long sheet of padding to be easily torn at uniform segments, such as every foot or every two feet. This can be accomplished by including in the die or roller pattern a flat horizontal space where there will be a concentration of perforations in a line, such that the layers will be securely bound together at that strip, but easily torn.

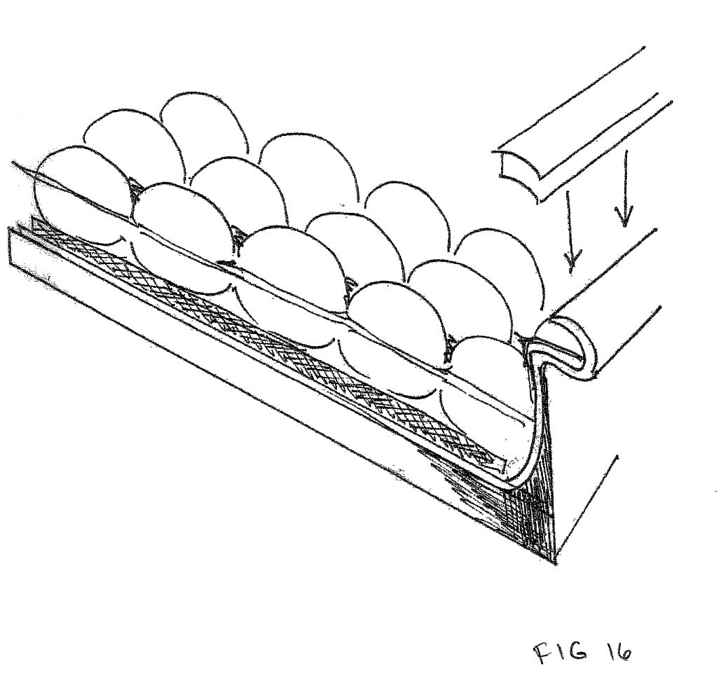

As shown in FIGS. 15 and 16, the resultant multiply layer is then inverted, and aligned on top of an absorbent layer 40 which is in turn aligned on top of a non-porous bottom layer 50 in a suitable forming die 70. The lateral edges of the forming die form a ledge and the lateral edges of the bottom layer extend beyond the absorbent layer, form a vertical surface and then extend onto the ledges of the forming die. The lips formed on the bubble layer also extends onto these ledges, and are bound to the lower layer by a heated die 72 or pair of dies lower thereupon. This forms a single strip of padding according to the present invention. The strips are preferable formed continuously many feet in length and then would onto rolls. The user, such as a hospital, would then mount the rolls horizontally (such as a horizontal dispenser for paper towels) and a desired length can be unrolled and cut or torn off.

While specific embodiments have been described in detail, those with ordinary skill in the art will appreciate that various modifications and alternatives to those details could be developed in light of the overall teachings of the disclosure. Accordingly, the particular arrangements disclosed are meant to be illustrative only and not limiting as to the scope of the invention, which is to be given the full breadth of any claims which are derivable from the description herein, and any and all equivalents thereof.

What is claimed is:

1. A disposable medical pad comprising:
a perforated top layer;
a perforated intermediate layer proximate to said top layer, said intermediate layer comprising a plurality of sealed and at least partially inflated chambers;
an absorbent layer proximate to said perforated intermediate layer; and
a bottom non-porous layer including upwardly extending opposing lateral edges.

2. The medical pad of claim 1 wherein said upwardly extending lateral edges are fixed to said intermediate layer.

3. The medical pad of claim 2 wherein said intermediate layer has outwardly extending lateral edges.

4. The medical pad of claim 2 wherein said upwardly extending lateral edges are fixed to said intermediate layer at said edge.

5. A disposable medical pad comprising:
a perforated top layer;
a perforated intermediate layer proximate to said top layer, said intermediate layer comprising a plurality of sealed and at least partially inflated chambers and having outwardly extending lateral edges located at approximately the midpoint of the height of said layer;
an absorbent layer proximate to said perforated intermediate layer; and
a bottom non-porous layer including upwardly extending opposing lateral edges.

6. A disposable medical pad comprising:
a perforated top layer;
a perforated intermediate layer proximate to said top layer, said intermediate layer comprising a plurality of sealed and at least partially inflated chambers, said chambers having contoured top and bottom surfaces forming pockets between proximate layers;
an absorbent layer proximate to said perforated intermediate layer; and
a bottom non-porous layer including upwardly extending opposing lateral edges.

7. A method for producing a multiple-layer sheet comprising the steps of:
forming uniform indentations in a first layer by contacting said first layer along its length against a roller having protrusions;
placing said first layer and two layers of thermoformable plastic between a pair of mating dies, bringing said dies into contact while introducing air between said layers of thermoformable plastic, applying heat through said dies to bind said layers of thermoformable plastic where said dies meet and creating sealed chambers of air;
perforating said first layer and said two layers of thermoformable plastic with a plurality of heated rods;
aligning said first layer and said two layers of thermoflamable plastic onto a layer of absorbent material above a wider thermoformable non-porous bottom layer; and
bending lateral edges of said bottom layer and heat bonding said lateral edges of said bottom to said two layers of thermoformable material.
